(19) United States
(12) Patent Application Publication
(10) Pub. No.: US 2016/0262631 A1
(43) Pub. Date: Sep. 15, 2016

(54) HANDSET MOBILE COMMUNICATION DEVICE FOR MEASURING BODY TEMPERATURE AND BODY TEMPRATURE MEASURING METHOD THEREOF

(71) Applicant: Ein-Yiao Shen, Taipei City (TW)

(72) Inventor: Ein-Yiao Shen, Taipei City (TW)

(21) Appl. No.: 14/743,388

(22) Filed: Jun. 18, 2015

(30) Foreign Application Priority Data
Mar. 12, 2015 (CN) 201510108485

(51) Int. Cl. A61B 5/01; A61B 5/00; G01J 5/02

(52) U.S. Cl. CPC A61B 5/015; G01J 5/0265; A61B 5/0077; A61B 5/746; A61B 5/6898; A61B 5/0035; A61B 5/7246; A61B 5/7282; G01J 2005/0077

(57) ABSTRACT

A handset mobile communication device for measuring body temperature and body temperature measuring method thereof is provided. The aforementioned handset communication device acquired an optical image and thermal image form an external target, then compared and correlated the two images and provided temperature information of selected portion of the target. Therefore, the aforementioned handset device may provide user to acquire temperature information of selected portion of target by contactless means. With respect to the previous contacting measurement, the device and method thereof of present application can provide more secure temperature measuring solution to health care professionals.

### BACKGROUND OF THE INVENTION

1. Field of the Invention

The present invention is related to a handset mobile communication device for measuring body temperature and body temperature measurement method. More particularly, the present invention is related to a measurement device and method thereof for remote measuring body temperature of target's selected portion.

2. Description of the Prior Art

In present body temperature measurement procedure, health care staff often use contact measurement means to measure patient's body temperature. After acquiring body temperature information, the staff need handwrite to record temperature information, or log in the medical management system to key in temperature information. When the health care staff need care a lot number of patients, measurement procedure would cause a heavy loading for them. In addition, when the patient suffering from highly contagious diseases, the contact measurement means will raise the infected risk of health care staff and seriously effects the safety of medical staff.

Furthermore, nowadays the human resource shortage problem is getting worse. If we can provide a measuring technique means which can rapidly measure automatically record patient's body temperature, then may reduce the medical staff's work loading.

In summary, providing a body measuring technical means which can raise the measuring speed is a technical issue need to be solved in the technical field.

### SUMMARY OF THE INVENTION

To solve the previous technical problems, one objective of the present application is providing a handset mobile communication device and measuring method thereof for remote measuring body temperature of selected portion of object.

To achieve the aforementioned objective, the present application provides a handset mobile communication device for measuring body temperature. The device comprises an image capturing module, a thermal measuring interface and a processor. Aforementioned image capturing module is configured to take an optical image from an external target. The thermal measuring interface is configured to electrically connect with an external thermal image sensor. The thermal image sensor is configured to sense the target and provide a thermal image related to the target. Processor is connected with the image capturing module and the thermal measuring interface. The processor further compares and correlates the optical image and the thermal image of the target so as to mark a selected portion of the target and provide a piece of temperature information of the selected portion.

To achieve the aforementioned objective, the present application provides a body temperature measuring method. The method is applied in a handset mobile communication device and comprises following steps: comparing and correlating an optical image and a thermal image of a target to mark a selected portion of the target, and providing a piece of temperature information of the selected portion.

Therefore, the handset mobile communication device the method thereof of present application acquires body temperature information of selected portion of target by comparing the optical image and thermal image so as to effectively speed up the temperature measuring procedure.

### BRIEF DESCRIPTION OF THE DRAWINGS

For a better understanding of the aforementioned embodiments of the invention as well as additional embodiments thereof, reference should be made to the Description of Embodiments below, in conjunction with the following drawings in which like reference numerals refer to corresponding parts throughout the figures.

FIG. 1 shows a block diagram of the handset mobile communication device for measuring body temperature of one embodiment of present application.

FIG. 2 shows a flow chart of the body temperature measuring method of one embodiment of present application.

FIG. 3 shows an operating schematic diagram of present application.

FIGS. 4-5 show schematic diagrams of then operating interface of the handset mobile communication device of present application.

FIG. 6 shows one embodiment of the thermal imaging sensor of present application.

FIGS. 7A and 7B show the other one embodiment of the thermal imaging sensor of present application.

### DETAILED DESCRIPTION OF THE PREFERRED EMBODIMENT

The following description is about embodiments of the present invention; however it is not intended to limit the scope of the present invention.

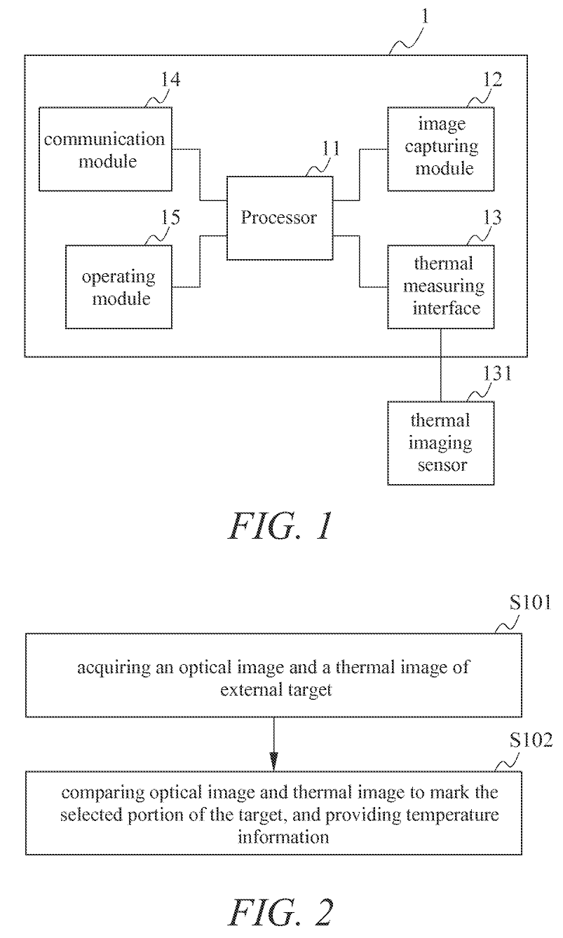

FIG. 1 shows a block diagram of handset mobile communication device for measuring body temperature. The handset mobile communication device 1 comprises a processor 11, image capturing module 12, thermal measuring interface 13, communication module 14, and operating module 15. The image capturing module 12 is configured to acquire an optical image from an external target. The thermal measuring interface 13 is configured to electrically connect with an external thermal imaging sensor 131 which is configured to acquire a thermal image from aforementioned target. The processor 11 further compares and correlates the optical image and the thermal image so as to provide a piece of temperature information of a selected portion of the target. The operating module 15 provides an operating interface which provides user operating the handset mobile communication device 1 of present application. The aforementioned operating interface can be implemented by software interface, hardware interface or touch controlling interface. The aforementioned thermal imaging sensor 131 can be implemented by the semiconductor thermal imaging sensor. The aforementioned image capturing module 12 comprises a camera and a control circuit thereof. The thermal measuring interface 13 comprises wireline connecting interface (e.g. physical plug, conductive line) or wireless connecting interface (e.g. RF communication interface, NFC communication interface). The communication module 14 comprises wireline communication circuit or wireless communication circuit (2G/3G/4G/5G, Wi-Fi, Bluetooth . . . etc.). Present application provides following two body temperature measuring means to achieve aforementioned objective, however the recognition mode is not limited by following description.

First recognition mode:

In another one embodiment, processor 11 recognizes the optical image so as to mark the selected portion, and identify the graphic feature of optical image and the thermal image (e.g. recognizing the graphic frame line of optical image and thermal image). Then, the processor 11 overlaps and compares two graphic frame lines which having the same or similar graphic feature to form a correlation. Next, the processor 11 recognizes the selected portion and related temperature information.

Second recognition mode:

In another one embodiment, the processor 11 compares and correlates each reference point of optical image and thermal image so as to generate a related correlation between the optical image and the thermal image. Then, the processor 11 recognizes the optical image to identify the selected portion of the target, and accesses temperature information of the selected portion of the thermal image related to the selected portion of the optical image.

In another embodiment, for quickly recording measured temperature information of the target, processor 11 further recognizes the optical image so as to provide a piece of identification (ID) information. The processor 11 further correlates ID information and temperature information to form a record file. The processor 11 may stores, or transmits this record file.

In another embodiment, the handset mobile communication device 1 of present application further comprises an alarm unit. The aforementioned alarm unit is connected with processor 11. The alarm unit further provides a piece of alarm information when temperature information exceeding the default value. The aforementioned alarm unit can be implemented by the software or hardware circuit.

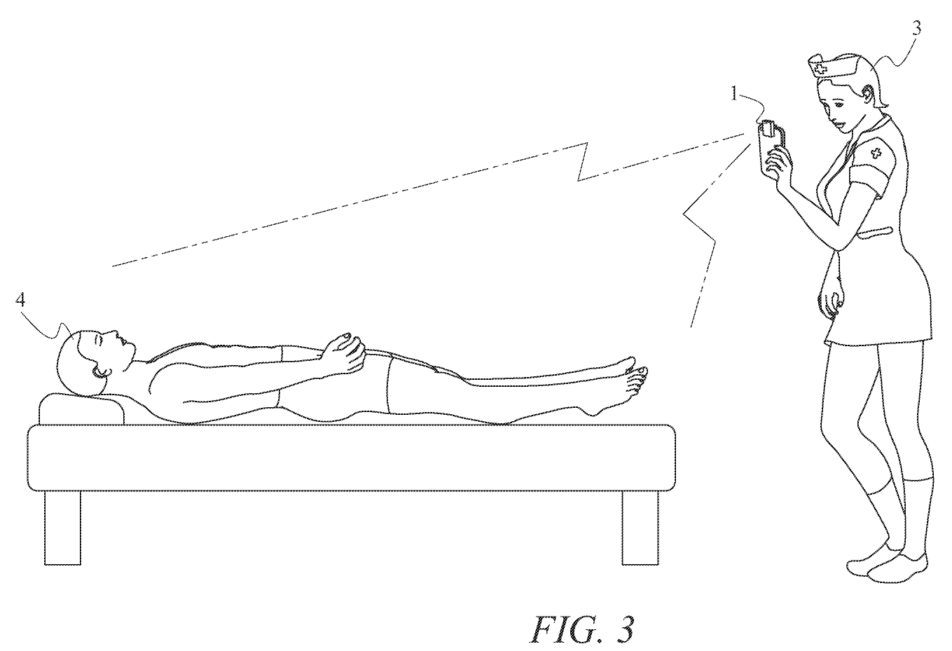

FIG. 2 shows a flow chart of body temperature measuring method of another one embodiment of present application. The steps of body temperature measuring method are descripted as below:

S101: acquiring an optical image and a thermal image of external target.

S102: comparing optical image and thermal image to mark the selected portion of the target, and providing temperature information.

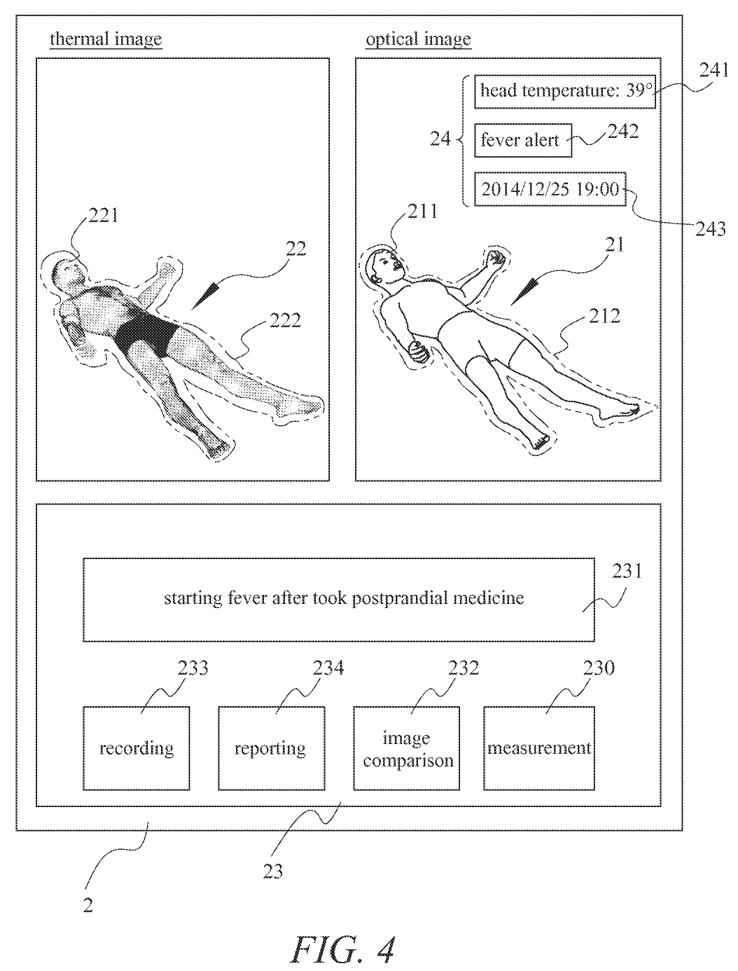

FIG. 3 shows an operating schematic diagram of present application. When user operates the handset mobile communication device 1 to shoot the target, then the device 1 acquires an optical image 21 and thermal image 22 of target. The image is displayed on the operating interface 2 of touch screen. The user may set the handset mobile communication device 1 to measure the temperature of selected portion (e.g. temperature of head).

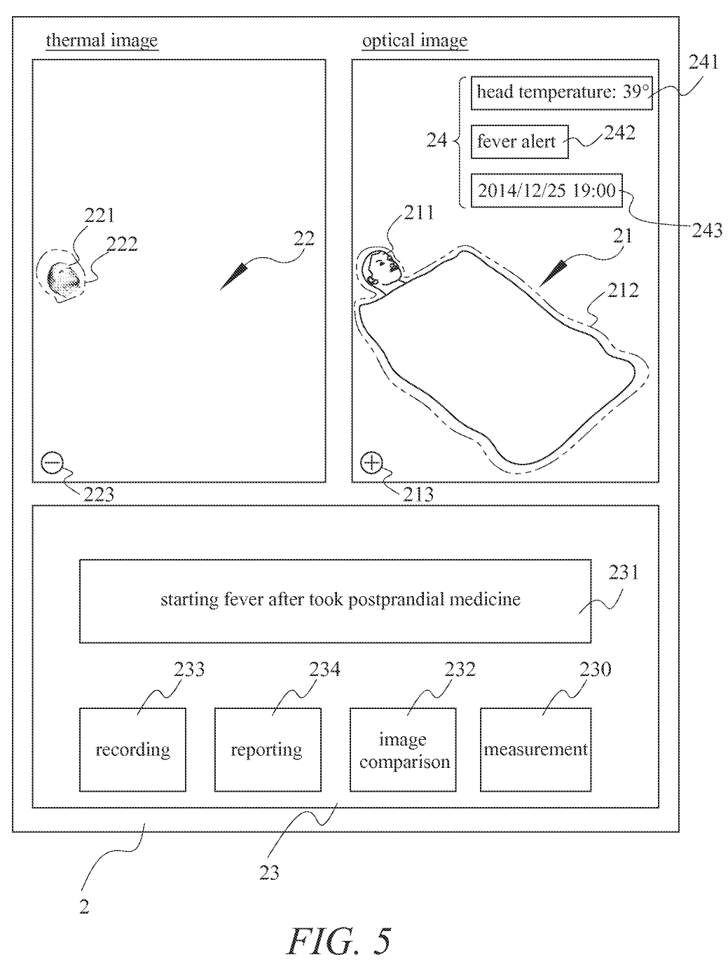

Please refer to the FIG. 4, when user triggers measurement button 230, it can drives the processor 11 acquiring the image of target 4. And after triggering image comparison button 232, the processor 11 may determine the graphic feature (e.g. distribution density of image pixel or image frame line) of two images whether same or similar to each other. Then the processor 11 selectively uses the first recognition mode or second recognition mode to measure the temperature of selected portion so as to provide measurement information. The aforementioned measurement information selectively comprises temperature information 241, alarm information or time information.

When the processor 11 determining the graphic feature of optical image and thermal image are same or similar to each other, then selects first recognition mode to identify the optical image 21. The first recognition mode can identify the selected portion (head) of optical image 21 by imaging recognition technique. Then, the processor 11 recognizes the graphic frame lines (212 and 222) of aforementioned images (21 and 22) and executes a similarity comparison. When the two frame line match a similarity determination, the processor 11 overlaps two images (21 and 22) to acquire the thermal image of head and related temperature information 241.

Please refer to the FIG. 5, when the target covered shelter (e.g. quilt), because of the processor 11 only acquires parts of thermal image of target 4, the processor 11 selects the second recognition mode to identify the temperature of head portion. When the processor 11 acquires the optical image 21 and thermal image 22, the processor 11 can base on the shooting angle or shooting position of the image capturing module 12 and thermal image sensor 13 to mark a reference point of each image (optical image reference point 213 and thermal image reference point 214). Next, the processor 11 builds a correlation by overlapping optical image reference point 213 and thermal image referencing point 223. The processor 11 further recognizes the optical image 21 to identify the selected portion (e.g. head portion) of the target 4 and acquires the selected portion 221 of thermal image 22 by the selected portion 211 of optical image 21 and aforementioned correlation.

Further explanation, processor 11 takes optical image reference point 213 as origin point(x₀, y₀). After the processor 11 identifies selected portion image from the optical image 21, then the processor 11 further calculates the correlation coordinate point(x₁, y₁) of selected portion. Next, the processor 11 further takes the thermal image reference point 223 as origin point(x₀, y₀), and acquires the related thermal image 22 and temperature information of the selected portion 221 based on the correlation between (x₀, y₀) and (x₁, y₁). When the processor 11 acquires temperature information of the selected portion 222, it may selectively operate a connection procedure which identifying the optical image 21 to provide identification (ID) information of target. For example, when the device 1 acquires optical image of patient' head or environment image (e.g. two dimension barcode, ward's doorplate, hospital bed's name card, patient's name card, patient's barcode bracelet) and identifies the image content to obtain ID information of target. Then processor 11 joints ID information and temperature information together to provide a record file. The format of record file is shown in table 1. User 3 may triggers recording button 233 to enable processor 11 store aforementioned record file, or transmitting the record file to a management device from communication module 14.

TABLE 1

| Identification result | Selected portion | Temperature information |
|---|---|---|
| David Lee | Head portion | 35.2° C. |

When the measured temperature information exceeds default value (e.g. 38° C.) then the processor 11 will generate alarm information. Furthermore, when the reporting button 234 is triggered, the processor 11 can transmit measurement information to assigned computer device by communication module 14 so as to let patient's family member acquire patient's situation immediately.

User 3 also uses the inserting area 231 of operating area 23 of operating interface 2 to insert patient's situation information, and merges situation information to the record file. The other format of record file is shown in table 2.

TABLE 2

| | Selected | Temperature | Measuring | Situation |
|---|---|---|---|---|
| ID | portion | information | time | information |
| David Lee | Head portion | 38.5° C. | 2014/12/25, | starting fever |
| | | | 19:00 | after took |
| | | | | postprandial |
| | | | | medicine |

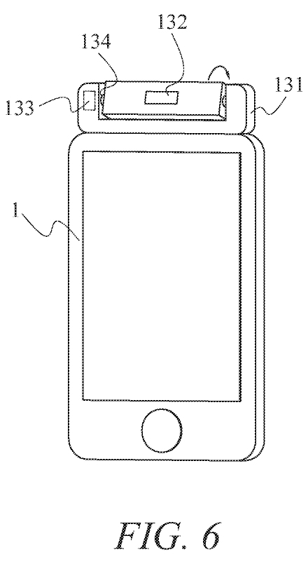

In another embodiment, the handset mobile communication device 1 of present application further provides a selfie operation. To achieve aforementioned operation, FIG. 6 shows one embodiment of the thermal imaging sensor 131. Thermal imaging sensor 131 comprises a rotator 133 and thermal sensor 132. The rotating end 134 of the rotator 133 connects with the thermal sensor 132, and portion body of the rotator 133 connects with the handset mobile communication device 1. The processor 11 further electrically connects with the thermal sensor 132 and rotator 133. When executing the selfie operation, the processor 11 enables the rotator 133 rotating so as to make the thermal sensor 132 faces to user and shoots a thermal image.

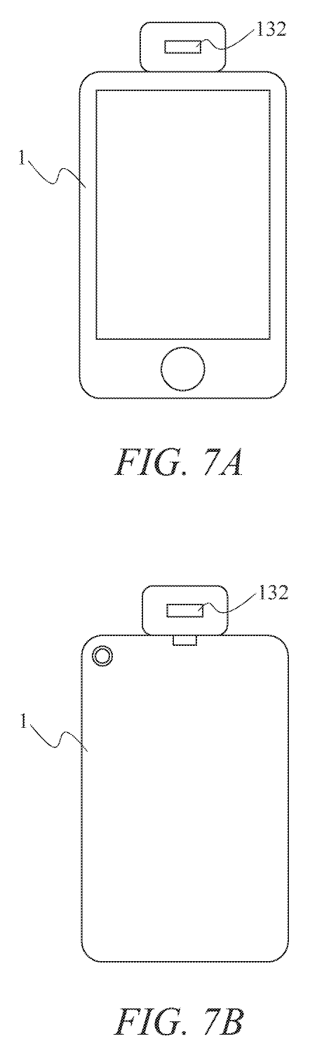

To achieve aforementioned selfie operation, FIGS. 7A and 7B show the other one embodiment of the thermal imaging sensor 131. Thermal imaging sensor 131 comprises two thermal sensors having two opposing shooting directions and electrically connecting with the processor 11. FIG. 7A and 7B shows a front view and back view of the handset mobile communication device 1 and the thermal imaging sensor 131 respectively. When executing the selfie operation, the processor 11 enables one thermal sensor (default: thermal sensor 132 of FIG. 7A) which facing the user, and shoots a thermal image from the target.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. A handset mobile communication device for measuring body temperature, comprising:
a measurement operation, comparing and correlating an optical image and a thermal image from an external target to mark a selected portion of the target and provide temperature information of the selected portion.

2. The device as claimed in claim 1, further comprising:
an image capturing module, configured to take the optical image from the external target;
an interface, configured to electrically connect with an external thermal imaging sensor, the thermal imaging sensor configured to measure the target and provide the thermal image related to the target; and
a processor, connected with the image capturing module and the interface, and configured to execute the measurement operation.

3. The device as claimed in claim 1, further generating a related correlation between the optical image and the thermal image based on a reference point of the optical image and the thermal image, recognize the optical image so as to identify the selected portion of the target, and provides the temperature information of the selected portion.

4. The device as claimed in claim 1, further recognizing the optical image so as to mark the selected portion, overlaps and compares the recognized optical image and the thermal image so as to identify the temperature information of the selected portion.

5. The device as claimed in claim 1, further recognizing the optical image so as to provide identify (ID) information of the target, and correlates the identify information and the temperature information to provide a record file.

6. The device as claimed in claim 2, further comprising an operating module connected with the processor, wherein the operating module further provides user inputting descripting information related to the temperature information.

7. The device as claimed in claim 1, further providing alarm information when the temperature information exceeds a default vale.

8. The device as claimed in claim 1, wherein the measurement operation further comprises a selfie operation.

9. The device as claimed in claim 2, wherein the thermal imaging sensor further comprises:
a thermal sensor, and
a rotator, electrically connected with the processor through the interface , a rotating end of the rotator connecting with the thermal sensor, and portion of the rotator connecting with body of the handset mobile communication device;
wherein, the processor further enables the rotator rotating the thermal sensor so as to set a shooting direction of the thermal sensor.

10. The device as claimed in claim 2, wherein the processor further operates a selfie operation to enable the rotator rotating the thermal sensor so as to face to a user.

11. The device as claimed in claim 2, wherein the thermal imaging sensor further comprises two thermal sensors having two opposing shooting directions.

12. The device as claimed in claim 11, wherein the processor further operates a selfie operation to enable one of the thermal sensor which faced to a user.

13. A body temperature measuring method, applied in a handset mobile communication device, comprising following steps:
comparing and correlating an optical image and a thermal image of a target so as to mark a selected portion of the target, and providing temperature information of the selected portion.

14. The method as claimed in claim 7, further generating a related correlation between the optical image and the thermal image based on a reference point of the optical image and the thermal image, and recognize the optical image so as to mark the selected portion of the target, and providing the related piece of temperature information of the selected portion.

15. The method as claimed in claim 7, further recognizing the optical image and the thermal image, overlapping and comparing the recognized optical image and the thermal image so as to provide the temperature information of the selected portion.

16. The method as claimed in claim 7, further recognizing the optical image to provide identify (ID) information of the target, and correlates the identify information and the temperature information to provide a record file.
